(19) United States
(12) Patent Application Publication
(10) Pub. No.: US 2019/0192059 A1
(43) Pub. Date: Jun. 27, 2019

(54) PHOTOACOUSTIC APPARATUS AND SIGNAL PROCESSING METHOD

(71) Applicant: CANON KABUSHIKI KAISHA, Tokyo (JP)

(72) Inventor: Kazuhiko Fukutani, Yokohama-shi (JP)

(21) Appl. No.: 16/329,573

(22) PCT Filed: Aug. 30, 2017
(86) PCT No.: PCT/JP2017/032011

§ 371 Date: Feb. 28, 2019

(30) Foreign Application Priority Data
Sep. 9, 2016 (JP) 2016-176057

(51) Int. Cl. A61B 5/145; A61B 5/00

(52) U.S. Cl. CPC A61B 5/14542; A61B 5/0095; A61B 5/72; A61B 5/7425; A61B 5/14546

(57) ABSTRACT

A photoacoustic apparatus of the present invention obtains distribution information of concentration of a substance inside an object using electric signals originated from acoustic waves generated from the object irradiated with a plurality of lights having a plurality of wavelengths, and includes a signal processor for obtaining a plurality of sound pressure distribution information, obtains concentration information of the substance at a certain position based on an user input, and obtains distribution information of the concentration of the substance inside the object using the sound pressure distribution information, concentration information of the substance, and information on absorption of each of the lights having the plurality of wavelengths by the substance.

### TECHNICAL FIELD

The present invention relates to a photoacoustic apparatus and a signal processing method.

### BACKGROUND ART

A photoacoustic imaging technique is an imaging technique that uses light. In photoacoustic imaging, an object is irradiated with pulsed light generated in the light source. The irradiated light propagates and diffuses in the object. When the energy of the irradiated light is absorbed by a light absorber inside the object, an acoustic wave (hereafter “photoacoustic wave”) is generated. By receiving this photoacoustic wave using a transducer, and analyzing and processing the received signals using a processor, information on optical characteristic values inside the object is obtained as image data. Thereby the characteristic value distribution related to light absorption inside the object (e.g. information distribution about light absorption of blood in blood vessels) can be visualized.

Further, distribution of the concentration of a substance (light absorber) that exists in the object can be determined by irradiating the object with lights having mutually different wavelengths. In particular, if the object is irradiated with lights having mutually different wavelengths, and information distribution about the light absorption of the blood in blood vessels at each wavelength is obtained, the concentration of oxyhemoglobin HbO and the concentration of deoxyhemoglobin Hb can be obtained, and oxygen saturation of the blood can be known. For example, when lights having two different wavelengths are used, the oxygen saturation distribution SO₂(r) is determined by the following Expression (1).

$$\begin{matrix} [ Math . 1 ] & \\ {SO}_{2} ( r ) = \frac{[ {Hb O}_{2} ]}{[ {Hb O}_{2} ] + [ Hb ]} = \frac{\frac{{μ}_{a}^{{λ}_{2}} ( r )}{{μ}_{a}^{{λ}_{1}} ( r )} \cdot {ɛ}_{Hb}^{{λ}_{1}} - {ɛ}_{Hb}^{{λ}_{2}}}{( {ɛ}_{HbO}^{{λ}_{2}} - {ɛ}_{Hb}^{{λ}_{2}} ) - \frac{{μ}_{a}^{{λ}_{2}} ( r )}{{μ}_{a}^{{λ}_{1}} ( r )} \cdot ( {ɛ}_{HbO}^{{λ}_{1}} - {ɛ}_{Hb}^{{λ}_{1}} )} & ( 1 )\end{matrix}$$

Here μₐ^{λ}^{₁}(r) denotes an absorption coefficient at wavelength λ₁ in a certain position r, and μₐ^{λ}^{₂}(r) denotes an absorption coefficient at a wavelength λ₂ in a certain position r. ε_{HbO}^{λ}^{₁} denotes a molar absorptivity of the oxyhemoglobin at the wavelength λ₁, and ε_{Hb}^{λ}^{₁} denotes a molar absorptivity of the deoxyhemoglobin at the wavelength λ₁. ε_{HbO}^{λ}^{₂} denotes a molar absorptivity of the oxyhemoglobin at the wavelength λ₂, and ε_{Hb}^{λ}^{₂} denotes a molar absorptivity of the deoxyhemoglobin at the wavelength λ₂, ε_{HbO}^{λ}^{₁}, ε_{Hb}^{λ}^{₁}, ε_{HbO}^{λ}^{₂}, ε_{Hb}^{λ}^{₂} are known values. r denotes a position coordinate. To determine the oxygen saturation of the blood, the ratio of absorption coefficients at two wavelengths is required, as shown in Expression (1).

The initial sound pressure distribution (P₀(r)) of the photoacoustic wave that is generated from the absorber inside the object by the light absorption is expressed by the following Expression (2).

[Math. 2]

P₀(r)=Γ(r)·μₐ(r)·Φ(r) (2)

Here Γ(r) is a Gruneisen coefficient at a certain position r, and is determined by dividing the product of the volume expansion coefficient (β) and a square of the sound velocity (c) by a specific heat at constant pressure (Cₚ), and normally depends on the position, but does not depend on the wavelength of the light. μₐ(r) denotes an absorption coefficient at a certain position r. Φ(r) denotes an intensity of light at a certain position r (an intensity of light irradiated to the absorber, also called “light fluence”). The initial sound pressure (P₀(r)) at a certain position r can be calculated using a received signal (PA signal) that is output from a probe which received the photoacoustic wave.

The value of the ratio of the absorption coefficients at two wavelengths can be determined as follows using Expression (2).

$$\begin{matrix}[ Math . 3 ] & \\ \frac{{μ}_{a}^{{λ}_{2}} ( r )}{{μ}_{a}^{{λ}_{1}} ( r )} = \frac{{Φ}^{{λ}_{1}} ( r ) \cdot {P}_{0}^{{λ}_{2}} ( r )}{{Φ}^{{λ}_{2}} ( r ) \cdot {P}_{0}^{{λ}_{1}} ( r )} = α \cdot \frac{{Φ}^{{λ}_{1}} ( r )}{{Φ}^{{λ}_{2}} ( r )} & ( 3 )\end{matrix}$$

As Expression (3) indicates, a coefficient α, which is a ratio of the intensity of light Φ^{λ}^{₁} at the wavelength λ₁, and the intensity of light Φ^{λ}^{₂} at the wavelength λ₂ must be determined to obtain the value of the ratio of absorption coefficients at a certain position r. PTL 1 discloses that a value, which can be expressed by a relational expression including an intensity of light at each wavelength, is regarded as the coefficient α, and the initial sound pressure P₀ is determined from the coefficient α and the received signal, whereby the oxygen saturation can be calculated.

### CITATION LIST

### Patent Literature

PTL 1: Japanese Patent Application Laid-Open No. 2015-205136

### SUMMARY OF INVENTION

### Technical Problem

In the case of the method according to PTL 1, the oxygen saturation can be calculated using one coefficient α at a certain position r or in regions where the light intensity ratio is substantially the same. However, α differs depending on the position once the distance from the light irradiation region on the surface of the object and the distance from the probe exceed the range where distance (depth) is regarded as uniform. Therefore in the case of a blood vessel or the like, which extends over regions that cannot be expressed by one coefficient α, oxygen saturation gradually changes in the display, even it is the same blood vessel (e.g. artery), in other words, calculation accuracy drops.

With the foregoing in view, it is an object of the present invention to obtain information on concentration easily and accurately in the photoacoustic measurement.

### Solution to Problem

The present invention provides a photoacoustic apparatus for obtaining distribution information of concentration of a substance inside an object, using electric signals originated from acoustic waves which are generated from the object irradiated with a plurality of lights having a plurality of wavelengths, respectively,

the photoacoustic apparatus comprising a signal processing unit configured to:

obtain a plurality of sound pressure distribution information corresponding to the plurality of wavelengths, respectively; and

obtain concentration information of the substance at a certain position inside the object which is determined based on an instruction from a user,

obtain the distribution information of the concentration of the substance inside the object, using the plurality of sound pressure distribution information, the concentration information of the substance at the certain position inside the object, and, information on absorption coefficient of the substance corresponding to each of the plurality of wavelengths.

The present invention also provides a photoacoustic apparatus for obtaining distribution information of concentration of a substance inside an object, using electric signals originated from acoustic waves which are generated from the object irradiated with a plurality of lights having a plurality of wavelengths, respectively,

the photoacoustic apparatus comprising a signal processing unit configured to:

obtain a plurality of sound pressure distribution information originated from the plurality of wavelengths, respectively;

obtain information indicating a difference of coefficients on attenuation of the lights having the plurality of wavelengths inside the object; and

obtain the distribution information of the concentration of the substance inside the object, using the plurality of pieces of sound pressure distribution information, the information indicating the difference, and information on absorption of each of the lights having the plurality of wavelengths by the substance.

The present invention also provides a signal processing method for obtaining distribution information of concentration of a substance inside an object, using electric signals originated from acoustic waves which are generated from the object irradiated with a plurality of lights having a plurality of wavelengths, respectively,

the signal processing method comprising:

a step of obtaining a plurality of sound pressure distribution information originated from the plurality of lights having the plurality of wavelengths, respectively, using the electric signals;

a step of obtaining concentration of the substance at a certain position inside the object; and

a step of obtaining the distribution information of the concentration of the substance inside the object, using the plurality of sound pressure distribution information, the concentration of the substance, and information on absorption of each of the plurality of lights having the plurality of wavelengths by the substance.

The present invention also provides a signal processing method that obtains distribution information of concentration of a substance inside an object using electric signals originated from acoustic waves which are generated from the object irradiated with a plurality of lights having a plurality of wavelengths, respectively,

the signal processing method comprising:

a step of obtaining a plurality of sound pressure distribution information originated from the plurality of lights having the plurality of wavelengths, respectively;

a step of obtaining information indicating a difference of coefficients on attenuation of the plurality of lights having the plurality of wavelengths inside the object; and

a step of obtaining the distribution information of the concentration of the substance inside the object, using the plurality of sound pressure distribution information, the information indicating the distance, and information on absorption of each of the plurality of lights having the plurality of wavelengths by the substance.

### Advantageous Effects of Invention

According to the present invention, information on concentration can be obtained easily and accurately in the photoacoustic measurement.

Further features of the present invention will become apparent from the following description of exemplary embodiments with reference to the attached drawings.

### BRIEF DESCRIPTION OF DRAWINGS

FIG. 1 is a schematic diagram depicting a general configuration of a photoacoustic apparatus to which Embodiment 1 can be applied.

FIGS. 2A and 2B are schematic diagrams depicting a signal processing unit to which Embodiment 1 can be applied.

FIG. 3 is a flow chart depicting an example of a processing flow of Embodiment 1.

FIGS. 4A to 4D are schematic diagrams depicting an example of a display screen of Embodiment 1.

FIG. 5 is a flow chart depicting an example of a processing flow of Embodiment 2.

FIG. 6 is a schematic diagram depicting an example of a display screen of Embodiment 2.

FIG. 7 is a flow chart depicting an example of a processing flow of Embodiment 3.

### DESCRIPTION OF EMBODIMENTS

Embodiments of the present invention will be described with reference to the drawings. Dimensions, materials, shapes of components, relative positions and the like thereof, which will be described below, should be changed appropriately depending on the configuration of the apparatus to which the invention is applied and on various conditions. Therefore the following description is not intended to limit the scope of the invention.

The present invention relates to a technique to detect an acoustic wave which propagates from an object, generate characteristic information inside the object, and obtain the information. Therefore the present invention can be regarded as an object information obtaining apparatus, or a control method thereof, or an object information obtaining method, or a signal processing method. Further, the present invention may be regarded as a program which causes an information processing apparatus, including such hardware resources as a CPU and memory, to execute these methods, or a storage medium storing this program. The storage medium may be a computer-readable non-transitory storage medium.

The object information obtaining apparatus of the present invention includes a photoacoustic imaging apparatus that utilizes the photoacoustic effect, the photoacoustic imaging apparatus receiving an acoustic wave, which is generated inside the object by irradiating the object with light (electromagnetic wave), and obtaining the characteristic information of the object as image data. In this case, the characteristic information is information on the characteristic values corresponding to a plurality of positions inside the object, respectively, and is generated using a received signal obtained by receiving the photoacoustic wave.

The characteristic information obtained by photoacoustic measurement is a value reflecting the absorptivity of light energy. For example, the characteristic information includes a generation source of an acoustic wave which is generated by irradiating light having a single wavelength, initial sound pressure inside the object, or light energy absorption density and absorption coefficient derived from the initial sound pressure. The concentration of a substance constituting a tissue can also be obtained from the characteristic information obtained by a plurality of mutually different wavelengths. If the oxyhemoglobin concentration and the deoxyhemoglobin concentration are determined as the substance concentrations, the oxygen saturation distribution information can be calculated. For the substance concentration, the total hemoglobin concentration, glucose concentration, collagen concentration, melanin concentration, volume fraction of fat and water and the like can be determined.

Based on the characteristic information obtained at a plurality of positions inside the object, two-dimensional or three-dimensional characteristic information distribution can be obtained. The distribution data can be generated as image data. The characteristic information may be determined, not as numeric data, but as distribution information at each position inside the object. The distribution information is, for example, an initial sound pressure distribution, an energy absorption density distribution, an absorption coefficient distribution, an oxygen saturation distribution or the like.

The acoustic wave that is referred to in the present invention is typically an ultrasound wave, including an elastic wave that is called a “sound wave”, or an “acoustic wave”. An electric signal which is converted from an acoustic wave by a transducer or the like is also called an “acoustic signal”. The phrases “ultrasound wave” and “acoustic wave” in this description are not intended to limit the wavelength of the elastic waves. An acoustic wave generated by the photoacoustic effect is called a “photoacoustic wave” or a “light-inducted ultrasound wave”. An electric signal originated from a “photoacoustic wave” is also called a “photoacoustic signal”.

In the following embodiments, a photoacoustic apparatus, which obtains distribution information of the light absorber inside an object by irradiating the object with pulsed light and receiving and analyzing the acoustic wave from the object based on the photoacoustic effect, will be described as the object information obtaining apparatus. The object is assumed to be a breast of a subject. The object, however, is not limited to a breast, and may be another segment, such as limbs of a subject, an animal, an inorganic object, a phantom or the like. The object information obtaining apparatus according to the following embodiments can suitably be used for diagnosing malignant tumors and vascular disease of humans and animals, and for follow up observation of chemotherapy.

### Embodiment 1

A configuration and processing of an object information obtaining apparatus according to Embodiment 1 will be described. In the drawings, as a rule the same composing elements are denoted with the same reference signs, where redundant description is omitted.

(General Apparatus Configuration)

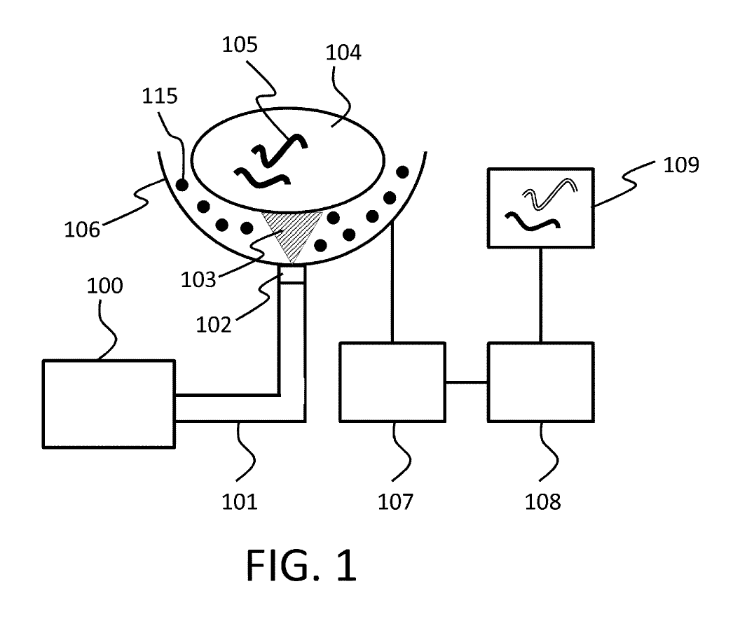

FIG. 1 is a schematic diagram depicting a configuration of a photoacoustic apparatus according to this embodiment. The photoacoustic apparatus of this embodiment has, at least: a light source 100, a probe 106 which includes a converting element 115 to receive a photoacoustic wave; and a signal processing unit 108 which obtains characteristic value information inside the object using received signals output from the converting element 115.

The light from the light source 100 is guided to a light emitting unit 102 by a light guiding unit 101, and is emitted from the light emitting unit 102. In the case of measuring oxygen saturation, the light source 100 outputs a plurality of pulsed lights having mutually different wavelengths at different timings. An irradiation light 103 emitted from the light emitting unit 102 is irradiated to an object 104, and reaches a light absorber 105, which is a target segment, inside the object. The light absorber 105 is typically a tumor in a living body, blood vessels, such a substance as hemoglobin that exists in blood vessels or the like. Each time the light absorber 105 absorbs the energy of respective lights having mutually different wavelengths, a photoacoustic wave is generated. The generated photoacoustic wave propagates through the object, and reaches a converting element 115.

Each of the plurality of converting elements 115 outputs a time series analog signal by receiving the photoacoustic wave. The output analog received signal is sent to a signal collecting unit 107 which amplifies an analog signal using an amplifier, and performs digital conversion using an AD converter, and is then input to a signal processing unit 108. A digital received signal (hereafter “received signal”) is sequentially input to the signal processing unit 108 for a number of irradiated pulsed light. The signal processing unit 108 generates characteristic value information inside the object using the input received signals. If the photoacoustic apparatus is a photoacoustic microscope or the like, a number of converting elements 115 of the probe may be 1. However, if the photoacoustic apparatus is an object information obtaining apparatus to inspect such objects as a breast, it is preferable that the probe 106 has a plurality of converting elements 115. Particularly it is preferable to three-dimensionally and densely arrange the plurality of converting elements 115 spherically, hemispherically or cylindrically, in order to increase the calculation accuracy of the characteristic information inside the object.

(Internal Configuration of Signal Processing Unit 108)

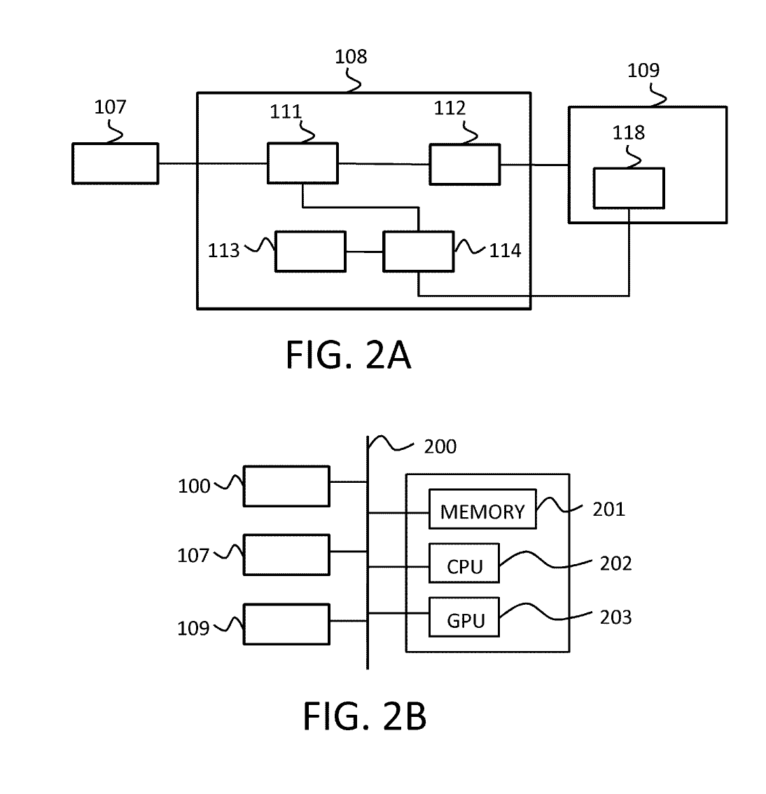

The configuration inside the signal processing unit 108 of this embodiment will be described next with reference to FIG. 2. FIG. 2A is a schematic diagram depicting a connection of a configuration (indicating detailed functions) inside the signal processing unit 108 of this embodiment and external configuration. FIG. 2B is a schematic diagram depicting a concrete configuration example of the signal processing unit 108. The signal processing unit 108 of this embodiment includes an information obtaining unit 111, a display control unit 112, a distance determining unit 113, and a coefficient determining unit 114.

The information obtaining unit 111 obtains the characteristic value information inside the object for each position, using the received signals output from the signal collecting unit 107. In concrete terms, data of the characteristic values corresponding to the positions on the two-dimensional or three-dimensional spatial coordinates (distribution data) by reconstructing the image using the time series received signals of each converting element 115. The unit region of the reconstruction is called a “pixel” or a “voxel”. For the image reconstruction method, a known image reconstruction method, such as Filtered Back Projection (FBP), time reversal method, model base method and Fourier transform method can be used. Delay and Sum Processing, which is used for ultrasound imaging, may be used.

In the case of a light focus type photoacoustic microscope, or a photoacoustic microscope using a focus type probe, distribution data may be generated without performing the image reconstruction processing. In concrete terms, the probe 106 and the light irradiation spot are relatively moved with respect to the object, using a scanning mechanism. The probe 106 receives the photoacoustic wave at a plurality of scanning positions. Then the information obtaining unit 111 performs the envelope detection for the obtained received signals with respect to the time change, converts the time axis direction of the received signals into the depth direction, and plots the received signals on the spatial coordinates. This is performed for each scanning position, whereby the distribution data can be configured.

The display control unit 112 generates image data to be displayed on the display unit 109, based on the characteristic information and the distribution data generated by the information obtaining unit 111. In concrete terms, based on the distribution data, the display control unit 112 performs such image processing as brightness conversion, distortion correction, extraction of a target region, blood vessel extraction processing, artery/vein separation processing, and logarithmic compression processing. Further, the display control unit 112 performs a control to display the distribution data along with various display items, and a control to update the display based on the instruction from an instruction unit 118 displayed on the display unit 109.

The distance determining unit 113 determines a distance d between the light irradiation region on the surface of the object and an arbitrary position (pixel or voxel) in the characteristic value information inside the object based on the shape information of the object and the light irradiation information. The distance d is used when the information obtaining unit 111 determines the characteristic value information based on the received signal. The distance d will be described in detail later, with reference to Expression (4).

The coefficient determining unit 114 determines the coefficient β which is used for the information obtaining unit 111 to determine the characteristic value information based on the received signals. The coefficient β will be described in detail later, with reference to Expression (6).

(Processing by Signal Processing Unit 108)

In this embodiment, as the characteristic value information, the information obtaining unit 111 determines at least the information on the sound pressure of the photoacoustic wave and the information on the oxygen saturation. In this description, “oxygen saturation” is an example of “information on concentration”, and indicates the ratio of hemoglobin combined with oxygen, out of the hemoglobin in red blood cells.

To determine the oxygen saturation, the ratio of the absorption coefficients at a plurality of wavelengths (at least two mutually different wavelengths), as shown in Expression (3), is required. The initial sound pressure (P₀) in Expression (3) indicates a relative value of the generated pressure of the photoacoustic wave actually generated in the object. Normally the light intensity distribution information Φ(r) can be simply expressed by the following Expression (4) using an analytic solution of the diffusion equation of an infinite medium.

[Math. 4]

Φ(r)=Φ₀ exp(−μ_{eff}·d(r)) (4)

Here Φ₀ is the light irradiation energy per unit area. μ_{eff} is an effective attenuation coefficient, and is given by μ_{eff}={3μa(μa+μs′)}1/2 where μs′ (reduced scattering coefficient) denotes the equivalent scattering coefficient of the object background, and μa denotes an absorption coefficient. d(r) denotes a distance between a certain position r inside the object and the light irradiation region on the surface of the object. Here the change in the effective attenuation coefficient, depending on the position, is negligibly small, and the effective attenuation coefficient of the object is assumed to be a coefficient which does not depend on the position inside the object.

Therefore Expression (3) is transformed to be the following Expression (5).

$$\begin{matrix}[ Math . 5 ] & \\ \frac{{μ}_{a}^{{λ}_{2}} ( r )}{{μ}_{a}^{{λ}_{1}} ( r )} = \frac{{Φ}^{{λ}_{2}} ( r ) \cdot {P}_{0}^{{λ}_{2}} ( r )}{{Φ}^{{λ}_{1}} ( r ) \cdot {P}_{0}^{{λ}_{1}} ( r )} = \frac{{Φ}_{0}^{{λ}_{2}} exp [ - {μ}_{eff}^{{λ}_{2}} \cdot d ( r ) ] \cdot {P}_{0}^{{λ}_{2}} ( r )}{{Φ}_{0}^{{λ}_{1}} exp [ - {μ}_{eff}^{{λ}_{1}} \cdot d ( r ) ] \cdot {P}_{0}^{{λ}_{1}} ( r )} & ( 5 )\end{matrix}$$

Here, if the irradiation energy Φ₀ per unit area is the same among the lights having mutually different wavelengths, then the following Expression (6) is established.

$$\begin{matrix}[ Math . 6 ] & \\ \frac{{μ}_{a}^{{λ}_{2}} ( r )}{{μ}_{a}^{{λ}_{1}} ( r )} = \frac{{P}_{0}^{{λ}_{2}} ( r )}{{P}_{0}^{{λ}_{1}} ( r )} exp [ ( {μ}_{eff}^{{λ}_{1}} - {μ}_{eff}^{{λ}_{2}} ) d ( r ) ] = \frac{{P}_{0}^{{λ}_{2}} ( r )}{{P}_{0}^{{λ}_{1}} ( r )} exp ( β \cdot d ( r ) ) & ( 6 )\end{matrix}$$

Here β is defined as β=μ_{eff}^{λ}^{₁}−μ_{eff}^{λ}^{₂}. In other words, the coefficient β is a difference of the effective attenuation coefficients between two mutually different wavelengths.

At this time, the oxygen saturation (SO₂) is given by the following Expression (7).

$$\begin{matrix}[ Math . 7 ] & \\ {SO}_{2} ( r ) = \frac{\frac{{P}_{0}^{{λ}_{2}} ( r )}{{P}_{0}^{{λ}_{1}} ( r )} \cdot exp [ β \cdot d ( r ) ] \cdot {ɛ}_{Hb}^{{λ}_{1}} - {ɛ}_{Hb}^{{λ}_{2}}}{( {ɛ}_{HbO}^{{λ}_{2}} - {ɛ}_{Hb}^{{λ}_{2}} ) - \frac{{P}_{0}^{{λ}_{2}} ( r )}{{P}_{0}^{{λ}_{1}} ( r )} \cdot exp [ β \cdot d ( r ) ] \cdot ( {ɛ}_{HbO}^{{λ}_{1}} - {ɛ}_{Hb}^{{λ}_{1}} )} & ( 7 )\end{matrix}$$

In this way, the oxygen saturation is approximately determined if: the ratio of the relative sound pressure distribution information (P₀(r)) of each wavelength that is obtained by calculation based on the received signal at each wavelength by the information obtaining unit 111; β which is a constant; and a distance d(r) between a certain position r inside the object and the light irradiation region on the surface of the object, are determined.

(Processing Flow)

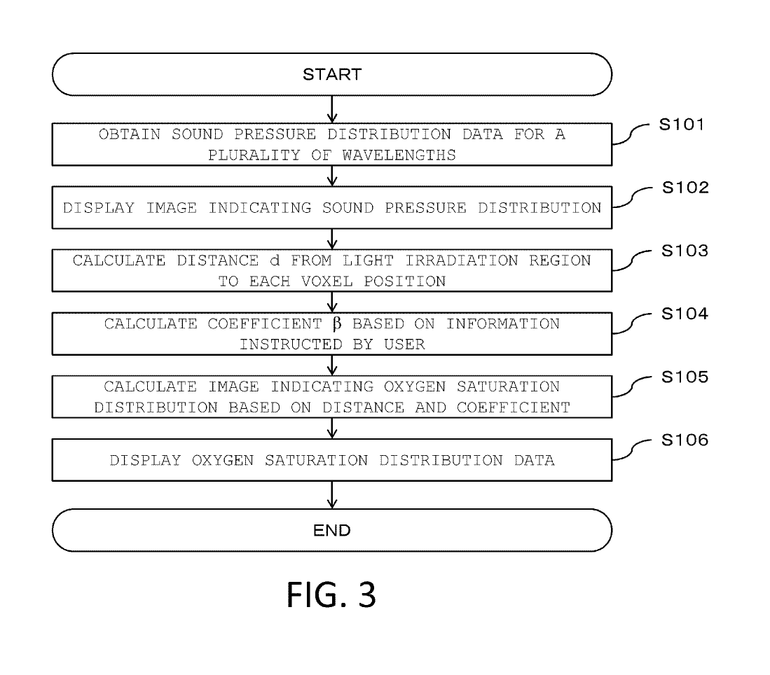

A processing flow when the signal processing unit 108 determines the oxygen saturation distribution will be described next. FIG. 3 is a flow chart depicting a processing to determine the oxygen saturation distribution according to this embodiment. The flow in FIG. 3 starts with the state after a received signal was sequentially input to the signal collecting unit 107 via the probe for each wavelength of the irradiated light, the signal collecting unit 107 performed such processing as AD conversion and amplification, and the received signal, converted into a digital signal, was sent to the signal processing unit 108.

In step S101, the information obtaining unit 111 obtains the sound pressure (P₀^{λ}^{₁}(r)) distribution data at the first wavelength λ₁ and the sound pressure (P₀^{λ}^{₂}(r)) distribution data at the second wavelength λ₂ using the input received signals.

In step S102, the display control unit 112 performs image processing based on the sound pressure distribution information for at least one wavelength, out of the sound pressure distribution information for a plurality of wavelengths, generated by the information obtaining unit 111, and displays an image indicating the sound distribution, or an image generated based on the image indicating the sound distribution, on the display unit 109. Examples of the image generated based on the image indicating the sound distribution are an image displaying only specific blood vessels, such as arteries or veins, an image of the difference or ratio between the images obtained at two wavelengths, and a pseudo-oxygen saturation distribution image.

In step S103, the distance determining unit 113 determines the distance d(r) from the light irradiation region on the surface of the object to the unit region (voxel or pixel) at the position r of the sound distribution data. The distance d is determined from the shape information of the object and the light irradiation information, for example. However, any method may be used as long as the distance d (r) from the light irradiation region on the surface of the object to the unit region at the position r of the sound distribution data can be determined.

The method of determining the shape information of the object is arbitrary. For example, the shape information may be determined by image processing from the sound pressure distribution data determined in step S101. Further, the shape information may be generated based on information of other measurement systems, such as an optical imaging apparatus, an ultrasound imaging apparatus, an MRI and CT. In the case of holding a breast with a cup type holding member, the shape of the object can be obtained based on the shape of the cup. The shape information may be calculated by the information obtaining unit 111, or may be input by the user to the information obtaining unit 111 in advance. The light irradiation information is such information as light irradiation energy distribution on the surface of the object, which is predetermined in the installation design. The light irradiation information may be obtained by the information obtaining unit 111 from the apparatus each time, or may be input by the user to the information obtaining unit 111.

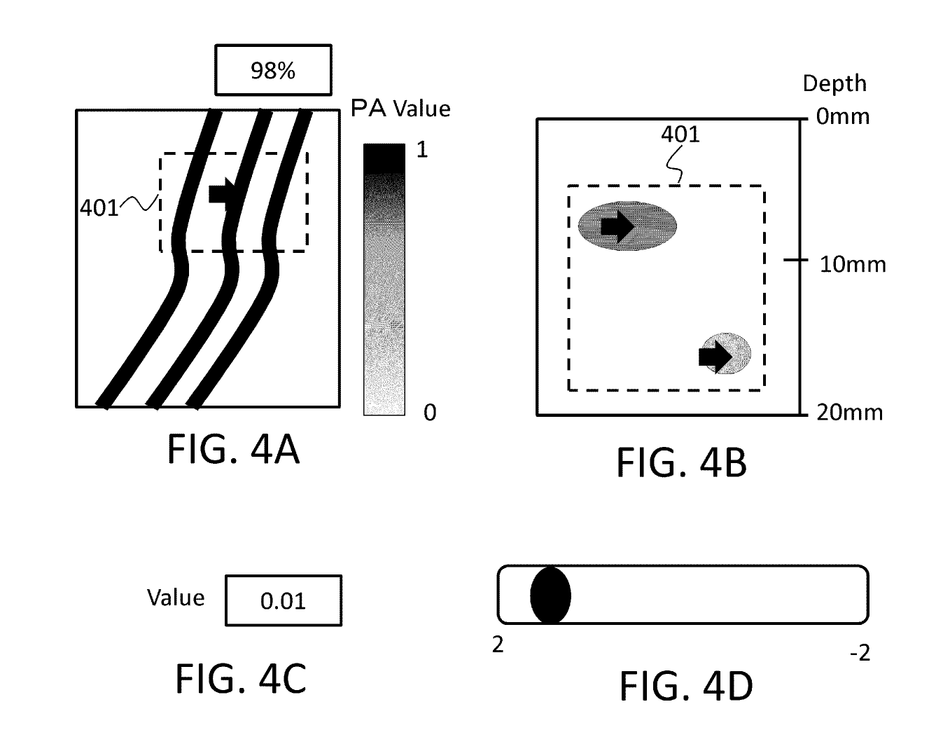

In step S104, the coefficient determining unit 114 determines a β value, which is a value of the coefficient β, via the instruction unit 118 on the display unit 109, based on biological information on the object instructed by the user. Here the biological information on the object input by the user is, for example, concentration information (specifically, the oxygen saturation value) at a position r selected by the user (indicated by the arrow mark in FIG. 4A) in the image displayed on the display unit in S102, and the coordinates of the position r as depicted in FIG. 4A. If the oxygen saturation value at the specified position r is known, the ratio of the absorption coefficients at two different wavelengths is determined. If the ratio of the absorption coefficients at two wavelengths is R when the oxygen saturation value is a certain value, then the coefficient β can be expressed by the following Expression (8) which is based on Expression (6).

$$\begin{matrix}[ Math . 8 ] & \\ β = \frac{1}{d ( r )} exp ( R \cdot \frac{{P}_{0}^{{λ}_{1}} ( r )}{{P}_{0}^{{λ}_{2}} ( r )} ) & ( 8 )\end{matrix}$$

In other words, the coefficient β can be determined from the absorption coefficient ratio R, the distance d (r) from the light irradiation region on the surface of the object to the pixel at the position r of the sound pressure distribution data determined in step S103, and the ratio of the sound pressure (P₀^{λ}^{₁}(r)) distribution data at the first wavelength λ₁ and the sound pressure (P₀^{λ}^{₂}(r)) distribution data at the second wavelength λ₂ determined in step S101.

It is preferable that the instruction unit 118 performs display to assist the user so that the user can easily instruct the biological information on the object. In concrete terms, as depicted in FIG. 4A, the display control unit 112 extracts an area where arteries and veins are clearly displayed, or an area where arteries and veins are accompanied, from the image displayed in step S102, and displays a guide frame 401. Thereby, the user can select a position of a blood vessel of which oxygen saturation is known, within the guide frame 401. If the user is guided by the instruction unit 118 in this way, the user can instruct the biological information without ambiguity.

The biological information on the object that is input by the user may be at least two position information on light absorbers, which have approximately the same absorption coefficients and are located at different depths, as depicted in FIG. 4B. Here the depth direction refers to an approximately vertical direction with respect to the light irradiation region. If the two positions, which are different at least in the depth direction, are r₁ and r₂ in the light absorbers which have approximately the same absorption coefficients and are located at different depths, then the coefficient β can be expressed by the following Expression (9) which is based on Expression (6).

$$\begin{matrix}[ Math . 9 ] & \\ β = \frac{1}{{r}_{2} - {r}_{1}} exp ( \frac{{P}_{0}^{{λ}_{1}} ( {r}_{2} )}{{P}_{0}^{{λ}_{1}} ( {r}_{1} )} - \frac{{P}_{0}^{{λ}_{2}} ( {r}_{2} )}{{P}_{0}^{{λ}_{2}} ( {r}_{1} )} ) & ( 9 )\end{matrix}$$

Here it is assumed that r₂>r₁. Using Expression (9), the coefficient β can be determined from the sound pressure (P₀^{λ}^{₁}(r)) distribution data at the first wavelength λ₁ and the sound pressure (P₀^{λ}^{₂}(r)) distribution data at the second wavelength λ₂ determined in step S101, and the position data r₁ and r₂. For the user to accurately indicate biological information on the object, it is preferable that the instruction unit 118 assist the user. In concrete terms, the display control unit 112 extracts an area of a same blood vessel, extending in the depth direction, in the image displayed in step S102, as depicted in FIG. 4B, and displays a guide frame 401. Thereby the user can select two points having different depths within the guide frame 401.

The coefficient determining unit 114 may receive an input of a value directly from the user using the instruction unit 118, as shown in FIG. 4C, and use the value as the coefficient β (that is, a difference of the effective attenuation coefficients at two mutually different wavelengths). When the coefficient β is received using the instruction unit 118, the numeric value may be directly input as shown in FIG. 4C, or may be input using a slide bar as illustrated in FIG. 4D.

In the case of displaying an assist UI indicated by the guide frame 401 in FIG. 4A, it is preferable to limit the range which the user can specify when input is received from the user. A region of the object where the method of approximately calculating the oxygen saturation according to this embodiment can be suitably applied is near the center of the light irradiation region, or a region facing this center region. This is because an approximate expression, Expression (4) for example, on attenuation of the intensity of light, may not always be established in the peripheral portions of the light irradiation region. Hence it is preferable that the display control unit 112 displays the guide frame 401 within a region where the approximation is established.

In step S105, the information obtaining unit 111 generates the oxygen saturation distribution data based on Expression (7) using: the coefficient β value determined by the coefficient determining unit 114; the distance d(r) from the light irradiation region on the surface of the object to an arbitrary voxel (or pixel) of the sound pressure distribution data determined by the distance determining unit 113; the sound pressure (P₀^{λ}^{₁}(r)) distribution data at the wavelength λ₁ and the sound pressure (P₀^{λ}^{₂}(r)) distribution data at the wavelength λ₂; and the known spectral information of oxy/deoxyhemoglobin. The oxygen saturation distribution data may be obtained not by Expression (7), but by another equivalent expression.

In step S106, the display control unit 112 generates image data based on the oxygen saturation distribution data generated by the information obtaining unit 111, and displays the image data on the display unit 109.

In this way, the user can obtain an image of the oxygen saturation distribution by instructing known biological information on the object to the signal processing unit 108 using the instruction unit 118, in the displayed image indicating the sound pressure distribution, or the image generated based on the image indicating the sound pressure distribution.

As described above, in this embodiment, the distance d from the light irradiation region and the coefficient β, which does not depend on the position, are used to determine the information on concentration, such as oxygen saturation. In other words, there is no need to use a coefficient value expressed by a relational expression including the intensity of light at each wavelength, as disclosed in PTL 1. As a result, the oxygen saturation distribution can be obtained accurately. Further, complicated computing that considered scattering and absorption is not required to calculate the light intensity distribution. Furthermore, in this embodiment, the β value is calculated from known biological information instructed by the user, therefore the image of the oxygen saturation distribution can be obtained using a simple method equivalent to PTL 1.

The concrete configuration of each composing block of the photoacoustic apparatus according to this embodiment will be described next.

(Signal Processing Unit 108)

For the information obtaining unit 111, such processors as a CPU and GPU (Graphics Processing Unit), and such an operational circuit as an FPGA (Field Programmable Gate Array) chip can be used. The information obtaining unit 111 may be constituted not by one processor or operational circuit, but by a plurality of processors and operational circuits.

The information obtaining unit 111 may includes a memory to store the received signals output from the signal collecting unit 107. The memory is typically constituted by a ROM, RAM and such a storage media as a hard disk. The memory may be constituted not by one storage media, but by a plurality of storage media.

In the same manner as with the information obtaining unit 111, the display control unit 112, the distance determining unit 113 and the coefficient determining unit 114 are constituted by combining one or more processor(s), such as a CPU and GPU, and one or more circuit(s), such as an FPGA chip. The display control unit 112, the distance determining unit 113 and the coefficient determining unit 114 may include a memory to store received signals, generated distribution data, display image data, various measurement parameters and the like. Memory is typically constituted by one or more ROM(s), RAM(s) and storage media such as a hard disk.

FIG. 2B depicts a relationship of a specific example of the signal processing unit 108 and an external configuration. In the case of FIG. 2B, the signal processing unit 108 includes a memory 201, a CPU 202 and a GPU 203.

The CPU 202 plays a part of the functions of the distance determining unit 113, the coefficient determining unit 114, and the display control unit 112 according to this embodiment. In concrete terms, the CPU 202 receives an instruction on various parameters and operations from the user via the instruction unit 118 on the display unit 109, and generates necessary control information, and controls each composing block via the system bus 200. The CPU 202 can also perform signal processing, such as integration processing and correction processing, for the digital signals stored in the memory 201. The CPU 202 also writes the processed digital signals in the memory 201 again, so that the digital signals can be used for generating the distribution data by the GPU 203.

The GPU 203 plays a part of the functions of the information obtaining unit 111, the display control unit 112, the distance determining unit 113, and the coefficient determining unit 114 according to this embodiment. In concrete terms, the GPU 203 creates distribution data using digital signals that are processed and written to the memory 201 by the CPU 202, and calculates the shape of the object. The GPU 203 also creates image data by applying various types of image processing, such as brightness conversion, distortion correction and extraction of a target region, to the created distribution data. The CPU 202 can also perform the same processing. For the signal processing unit depicted in FIG. 2B, a PC or workstation, in which the CPU executes the operation specified in each step of the predetermined information processing according to the program developed in memory, is suitable.

(Light Source 100)

For the light source 100, a pulse light source, which can generate pulsed light in the nanosecond to micro second order, is preferable. 1 to 100 nanosecond(s) is desirable as the pulse width for actual use. For the wavelength, a wavelength in the 400 nm to 1600 nm range is used. To image a deep part of a living body in particular, a light having a wavelength which is absorbed by a specific inspection target substance (e.g. hemoglobin), out of the components constituting a living body, and which is not absorbed very much by other substances, is used. In concrete terms, a wavelength in the 700 nm to 1100 nm range is preferable. To image blood vessels near the surface of a living body at high resolution, on the other hand, using a wavelength in the visible light region is preferable. However, a wavelength in the tetra hertz, micro and radio wave regions can be used.

In concrete terms, laser is preferable as the light source 100. In this embodiment, which has lights having a plurality of wavelengths, a laser which can convert a wavelength to be oscillated is ideal. However, it is also possible to use a plurality of laser units which oscillate lights having mutually different wavelengths while switching oscillation. In the case of using a plurality of laser units, these laser units are regarded as one light source in this description.

For the laser, various lasers, including a solid-state laser, a gas laser, a dye laser and a semiconductor laser can be used. Particularly, such a pulse laser as an Nd:YAG laser and an alexandrite laser is preferable. A Ti:sa laser and an OPO (Optical Parametric Oscillator) laser, which uses an Nd:YAG laser light as the excitation light, may be used. And a light emitting diode, a flash lamp or the like may be used instead of a laser.

(Light Guiding Unit 101, Light Emitting Unit 102)

The light guiding unit 101 and the light emitting unit 102 transfer light from the light source 100 to the object 104. For the light guiding unit 101 and the light emitting unit 102, such an optical element as a lens, a mirror and an optical fiber can be used. However, the object may be irradiated with light directly from the light source 100. In the case of a biological information obtaining apparatus for inspecting a breast or the like, it is preferable that the light emitting unit 102 widens the diameter of the beam using a lens or the like, and then irradiate the light. In the case of a photoacoustic microscope, on the other hand, it is preferable to focus the diameter of the beam using a lens or the like, in order to increase the resolution, and then irradiate the light. The light emitting unit 102 may be movable with respect to the object 104, thereby a wide range of the object 104 can be imaged.

(Probe 106)

The probe 106 has one or more converting elements 115. For the converting elements 115, any converting element that can receive an acoustic wave and convert the acoustic wave into an electric signal can be used, including a piezoelectric element using a piezoelectric phenomena of lead zirconate titanate (PZT) or the like, a converting element using the resonance of light, and a capacitance type converting element such as CMUT. In the case of including a plurality of converting element 115, it is preferable that the converting elements are disposed on a plane or curved surface in an arrangement called a 1D array, a 1.5D array, a 1.75D array, a 2D array, an arc array or a hemispheric array.

In the case of a biological information obtaining apparatus to inspect a breast or the like, it is preferable that the probe 106 can mechanically move with respect to the object, in order to image a wide range. In the case of a handheld probe 106, the user may hold and move the probe 106. In the case of a photoacoustic microscope, it is preferable that the probe 106 is a focus type probe, and it is also preferable that the probe 106 can mechanically move along the surface of the object 104. It is also preferable that the irradiation position of the irradiation light 103 and the probe 106 move synchronously. An amplifier for amplifying an analog signal output from the converting element 115 may be disposed in the probe 106.

(Display Unit 109)

For the display unit 109, such a display as an LCD (Liquid Crystal Display), a CRT (Cathode Ray Tube) and an organic EL display can be used. The display unit 109 may be provided standalone and connected to the photoacoustic apparatus, instead of being embedded in the photoacoustic apparatus of this embodiment.

(Instruction Unit 118)

The instruction unit 118 is constituted by an input unit by the user and a guide, via an image or a sound, to instruct the method of input. For the input unit, a mouse, a keyboard, a touch panel, a voice input unit or the like can be used. The instruction unit 118 may be provided standalone and connected to the photoacoustic apparatus, instead of being embedded in the photoacoustic apparatus of this embodiment.

(Object 104)

Although the object 104 is not a part of the photoacoustic apparatus, it will be described below. The photoacoustic apparatus according to this embodiment may be used for diagnosing malignant tumors and vascular diseases of humans and animals, and for follow up observation of chemotherapy. Therefore the object 104 is assumed to be a living body, specifically a diagnostic target segment such as a breast, neck and abdomen of humans and animals. For example, if the measurement target is a human body, the target of the light absorber 105 may be oxyhemoglobin, deoxyhemoglobin, blood vessels which contain a high concentration of oxy(deoxy)hemoglobin, or new blood vessels that are generated near a tumor.

### Embodiment 2

Embodiment 2 will be described next. A photoacoustic apparatus of this embodiment has the same configuration as the photoacoustic apparatus of Embodiment 1, hence a detailed description of each component will be omitted. In the following description, the processing content of the signal processing unit 108, which is different from Embodiment 1, will be primarily described.

(Processing Flow)

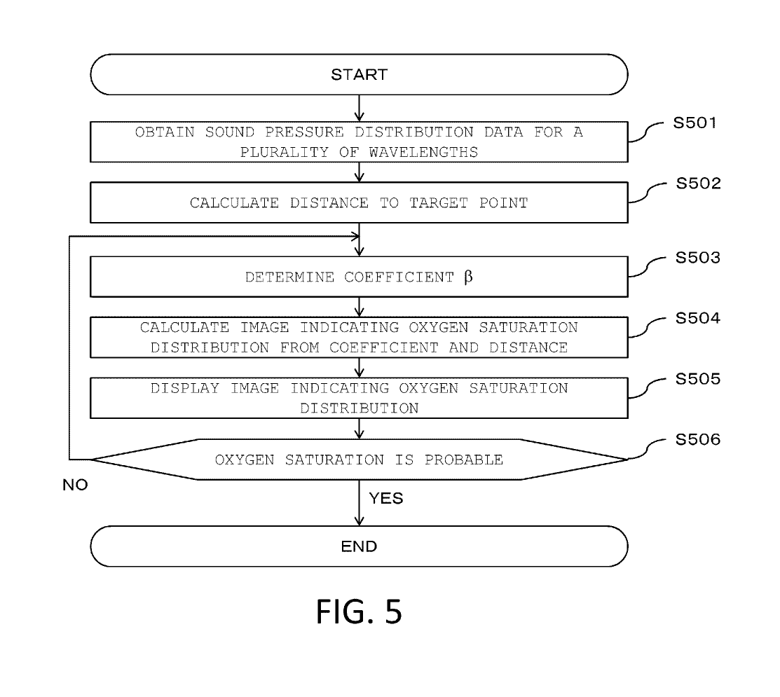

A processing flow of the signal processing unit 108 of this embodiment, to determine the oxygen saturation distribution, will be described next with reference to FIG. 5. The flow in FIG. 5 starts with the state after the received signals were sequentially input to the signal collecting unit 107 from the probe 106 for each wavelength of the irradiated light, the signal collecting unit 107 performed such processing as AD conversion and amplification, and then the received signal, converted into a digital signal, was sent to the signal processing unit 108.

In step S501, the information obtaining unit 111 obtains the sound pressure (P₀^{λ}^{₁}(r)) distribution data at the wavelength λ₁ and the sound pressure (P₀^{λ}^{₂}(r)) distribution data at the wavelength λ₂ using the input received signals.

In step S502, the distance determining unit 113 determines the distance d(r) from the light irradiation region on the surface of the object to a unit region at a position r in the sound pressure distribution data. The distance d can be determined from the shape information of the object and the light irradiation information, for example, as in S103 of Embodiment 1.

In step S503, the coefficient determining unit 114 receives information on the β value which the user input using the instruction unit 118 on the display unit 109, and instructs the β value to the information obtaining unit 111. In this stage, the user may input an arbitrary value for the β value. The user may input the β value itself, as shown in FIG. 4C, or may input the β value using a slide bar or the like, as shown in FIG. 4D. In the former case, the β value may be determined by inputting a β value which the information obtaining unit 111 appropriately assumed, or a predetermined β value, instead of a β value input by the user.

In step S504, the information obtaining unit 111 obtains the oxygen saturation distribution data using: the instructed coefficient β value; the distance d(r) calculated in step S502; the sound pressure (P₀^{λ}^{₁}(r)) distribution data at the wavelength λ₁ and the sound pressure (P₀^{λ}^{₂}(r)) distribution data at the wavelength λ₂; and Expression (7). Instead of Expression (7), another equivalent expression may be used to obtain the oxygen saturation distribution data.

In step S505, the display control unit 112 generates image data based on the oxygen saturation distribution data generated by the information obtaining unit 111, and displays the image data on the display unit 109. Then the user can confirm whether the oxygen saturation distribution is probable by viewing the displayed image of the oxygen saturation distribution. An example of a method for determining whether the oxygen saturation is probable is determining a blood vessel position of an artery based on the image of the sound pressure distribution displayed in step S502, and determining that the oxygen saturation is probable if the oxygen saturation is likely if the oxygen saturation in the blood vessel position is a value of around 95%. If there is accompanying blood vessels where an artery and vein run side by side, it may be determined whether the oxygen saturation is correct by focusing on the accompanying blood vessels. The user inputs the determination result using the instruction unit. Instead of receiving the determination result from the user, this determination may be performed by image recognition.

In step S506, the determination information input by the user is determined. If the determination result is YES (“oxygen saturation is probable”), processing ends, and if the determination result is NO (“oxygen saturation is not probable”), processing returns to step S503.

If processing returns to step S503, the coefficient determining unit 114 receives input of the changed β value from the user again, and the oxygen distribution data based on the changed β value is generated in step S504. In step S505, based on the new oxygen saturation distribution data, the image of the oxygen saturation distribution before changing the β value is changed (updated) to the image of the oxygen saturation distribution after changing the β value. This processing is repeated until the user inputs the determination information indicating that “oxygen saturation is probable”.

The second or later input of the β value in step S503 and the input of the determination information that results in NO (“oxygen saturation is not probable”) in step S506 may be performed all at once. In other words, if a β value that is different from the β value previously input ((n−1)th execution of step S506) is received in the nth execution of step S506, this may be regarded as NO in step S506. In this case, step S503 in the subsequent ((n+1)th time) of execution can be omitted. If the change in the β value is not instructed in step S506, the determination result is “oxygen saturation is probable”, hence processing flow ends.

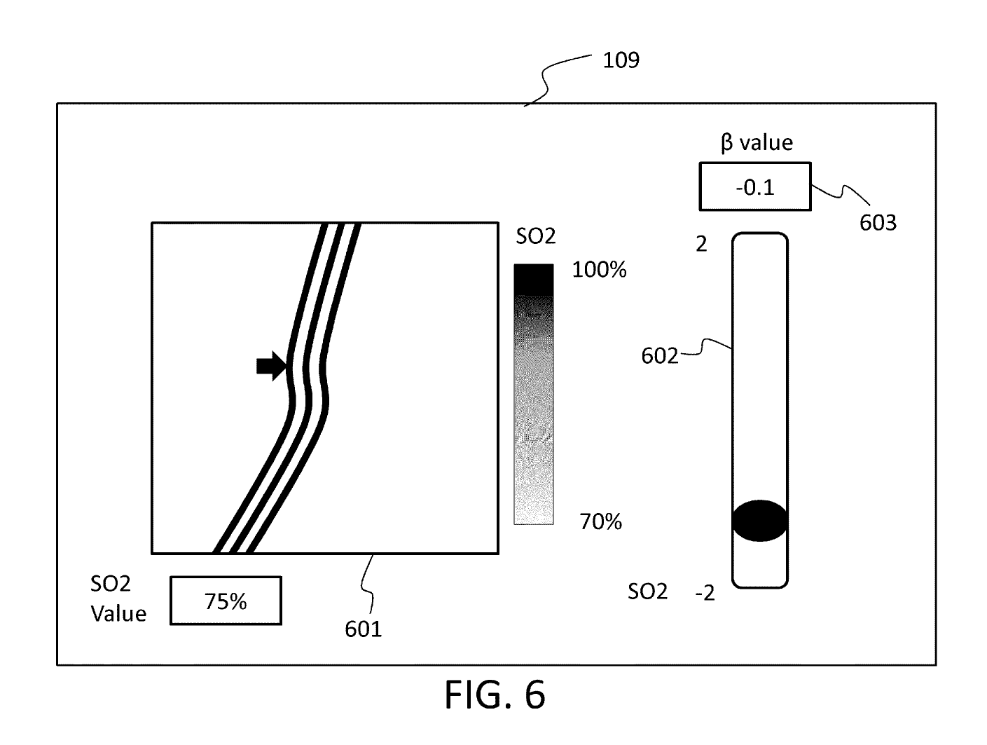

FIG. 6 is a schematic diagram depicting an example of the display screen and the instruction unit 118 which are displayed on the display unit 109 in step S505. The reference sign 601 indicates an image of the oxygen saturation distribution which was obtained using the β value and the distance d. An item for the guide display, to indicate the relationship between the brightness value and the oxygen saturation, is displayed next to the image of the oxygen saturation distribution. The value of the oxygen saturation at a position may be displayed by the user moving the cursor on the image of the oxygen saturation distribution, and specifying the arbitrary target position (e.g. artery). By displaying the value of the oxygen saturation at the target position like this, visibility when the user determines whether the oxygen saturation is probable or not improves.

The reference sign 602 indicates the instruction unit 118, and is a slide bar for the user to input the β value. If the user slides this slide bar, the β value, determined by the coefficient determining unit 114, is changed. As the β value is changed, the image of the oxygen saturation distribution is updated accordingly. As a display item to input the β value, a frame to directly input the value, as indicated by the reference sign 603, may be used.

As described above, according to this embodiment, the β value which the user directly input is used to determine the information on concentration, such as the oxygen saturation. Thereby the oxygen saturation distribution can be easily obtained. In this embodiment, an even more accurate image of the oxygen saturation distribution can be obtained by updating the β value.

In the above mentioned processing flow, information on the β value is input by the user, and the coefficient determining unit 114 determines the β value based on this input information, and instructs the β value to the information obtaining unit 111, but this embodiment is not limited to this method. In other words, the coefficient determining unit 114 may instruct a β value to the information obtaining unit 111, even if a β value is not input by the user.

For example, since it is not necessary to input a correct β value from the beginning, the coefficient determining unit 114 instructs a predetermined β value in the first execution of step S503. Then the coefficient determining unit 114 repeats the instruction to the information obtaining unit 111 while changing the value of the β value little by little, until the user inputs the determination information that indicates that the oxygen saturation distribution is probable. In such a configuration as well, the oxygen saturation distribution can be simply obtained. Further, the user may input target person information on age and race of the subject (person to be examined). Based on such an input, the coefficient determining unit 114 may obtain a β value which is statistically derived from this target person information in the first execution of step S503, and instruct the β value. It is preferable to use this kind of target person information since a probable oxygen saturation can be efficiently obtained.

### Embodiment 3

Embodiment 3 will be described next. A photoacoustic apparatus of this embodiment has the same configuration as the photoacoustic apparatus of Embodiment 1, hence a detailed description of each component will be omitted. In the following description, the processing content of the signal processing unit 108, that is different from Embodiment 1, will be primarily described.

(Processing Flow)

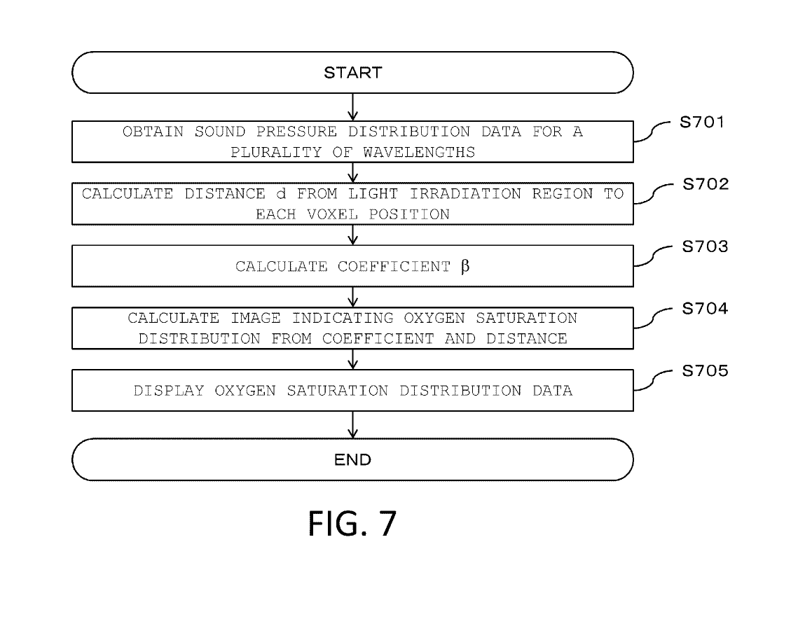

A processing flow of the signal processing unit 108 of this embodiment, to determine the oxygen saturation distribution, will be described next with reference to FIG. 7. The flow in FIG. 7 starts with the state after the received signals were sequentially input to the signal collecting unit 107 from the probe 106 for each wavelength of the irradiated light, the signal collecting unit 107 performed such processing as AD conversion and amplification, and the received signal, converted into a digital signal, was sent to the signal processing unit 108.

In step S701, the information obtaining unit 111 obtains the sound pressure (P₀^{λ}^{₁}(r)) distribution data at the wavelength λ₁ and the sound pressure (P₀^{λ}^{₂}(r)) distribution data at the wavelength λ₂ using the input received signals.

In step S702, the distance determining unit 113 determines the distance d(r) from the light irradiation region on the surface of the object to a unit region at a position r of the sound pressure distribution data. The distance d can be determined from the shape information of the object and the light irradiation information, for example, as in step S103 of Embodiment 1.

In step S703, the coefficient determining unit 114 determines the β value based on a known biological information on the object. The signal processing unit 108 performs image processing on the image generated in step S701, and extracts a position of an artery or vein determined based on the biological information, for example. In concrete terms, if the shape of the target blood vessel, such as an artery, is known, the position of the target blood vessel can be automatically specified using the pattern matching method. Another possible method is calculating a Hessian of an image, which is used for extracting a blood vessel in CT, and regarding the cylindrical structure as a blood vessel. Any other blood vessel extraction method may be used.

In the case of using a pattern matching method, the coefficient determining unit 114 obtains template data, which indicates the shape of the target blood vessel, from the storage unit or the like. The template data can be created by simulation or by actual measurement. If the data of an image in the sound pressure distribution is similar to this template data, it is assumed that this image is more probable to be the target blood vessel. Therefore the coefficient determining unit 114 extracts a part of the sound pressure distribution, and calculates the similarities with the template data. The coefficient determining unit 114 repeats the similarity calculation while shifting the portion to be extracted from the sound pressure distribution, whereby the position of which similarity is higher than a predetermined threshold is determined. As a result, an image similar to the template data (that is, the target blood vessel) can be extracted. The similarity can be calculated by Zero-mean Normalized Cross-Correlation (ZNCC). The parameters which indicate similarity, such as SSD (Sum of Squared Difference) and SAD (Sum of Absolute Difference) may be used.

The oxygen saturation of the artery or vein extracted by the above mentioned method or the like is often known biologically. Therefore based on the known information, the oxygen saturation value of the extracted artery or vein at a certain position r can be determined. If the oxygen saturation value at a certain position r is determined, the coefficient determining unit 114 can automatically determine the β value from: the sound pressure (P₀^{λ}^{₁}(r)) distribution data at the wavelength λ₁ and the sound pressure (P₀^{λ}^{₂}(r)) distribution data at the wavelength λ₂ determined in step S701; and the distance d(r) from the light irradiation region on the surface of the object to the unit region at the position r of the sound pressure distribution data determined in step S702, using Expression (8).

The coefficient determining unit 114 may determine the β value by another method. For example, pixels which are likely the blood vessel are extracted from the image generated in step S701, and if one blood vessel extends in the depth direction from the light irradiation region, then the different positions r₁ and r₂ of the blood vessel in the depth direction can be obtained. In this case, the coefficient determining unit 114 can automatically obtain the β value from the sound pressure (P₀^{λ}^{₁}(r)) distribution data at the wavelength λ₁ determined in step S701 and r₁ and r₂ using Expression (9).

In step S704, the information obtaining unit 111 generates the oxygen saturation distribution data from: the coefficient β value determined by the coefficient determining unit 114; the distance d(r) from the light irradiation region on the surface of the object to an arbitrary unit region (voxel or pixel) of the sound pressure distribution data determined by the distance determining unit 113; the sound pressure (PA^{λ}^{₁}(r)) distribution data at the wavelength λ₁; and the sound pressure (PA^{λ}^{₂}(r)) distribution data at the wavelength λ₂, using Expression (7).

In step S705, the display control unit 112 generates image data based on the oxygen saturation distribution data generated by the information obtaining unit 111, and displays the image data on the display unit 109.

Thereby, the signal processing unit 108 can automatically calculate the β value from known biological information, and obtain an image of the oxygen saturation distribution.

As described above, in this embodiment, the information obtaining unit 111 automatically calculates and determines a value when information on such concentration as the oxygen saturation is determined. Thereby the oxygen saturation distribution can easily be obtained without depending on an instruction by the user.

According to each embodiment of the present invention, when the oxygen saturation distribution is determined using the characteristic value information distribution originated from the photoacoustic wave generated by irradiating the object with lights with a plurality of wavelengths in the photoacoustic measurement, the information can be obtained easily and accurately, without determining the light intensity distribution for each wavelength inside the object. As a result, the present invention is effective to simplify an otherwise complicated computing on light propagation, to reduce computing cost, and to improve a real-time operation.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-176057, filed on Sep. 9, 2016, which is hereby incorporated by reference herein in its entirety.

1-16. (canceled)

17. A photoacoustic apparatus for obtaining distribution information of oxygen saturation inside an object, using electric signals originated from acoustic waves which are generated from the object irradiated with a plurality of lights having a plurality of wavelengths, respectively,
the photoacoustic apparatus comprising a signal processing unit configured to:
obtain a plurality of sound pressure distribution information corresponding to the plurality of wavelengths, respectively;
obtain position information on a position of an artery or vein inside the object, and
obtain oxygen saturation at the position of the artery or vein inside the object which is represented by the position information,
obtain the distribution information of the oxygen saturation in a region other than the artery or vein inside the object, using the plurality of sound pressure distribution information, the oxygen saturation at the position of the artery or vein inside the object, and information on absorption coefficient of oxyhemoglobin and de-oxyhemoglobin corresponding to each of the plurality of wavelengths.

18. The photoacoustic apparatus according to claim 17, wherein the signal processing unit is configured to obtain the position information on the position of the artery or vein which is instructed by an user.

19. The photoacoustic apparatus according to claim 17, wherein the signal processing unit is configured to obtain the position information on the position of the artery or vein by performing image recognition.

20. The photoacoustic apparatus according to claim 17, wherein the signal processing unit is configured to obtain the position information on a plurality of positions located at different depths inside the object as the position of the artery or vein.

21. The photoacoustic apparatus according to claim 17, wherein the signal processing unit is configured to obtain effective attenuation coefficient of each of the plurality of lights having the plurality of wavelengths inside the object based on the oxygen saturation at the position of the artery or vein inside the object, and
further obtain the distribution information of the oxygen saturation in the region other than the artery or vein inside the object using the effective attenuation coefficient.

22. The photoacoustic apparatus according to claim 21, wherein the signal processing unit is configured to obtain the distribution information of the oxygen saturation in the region other than the artery or vein inside the object using a difference between the effective attenuation coefficient of each of the plurality of lights corresponding to the plurality of wavelengths.

23. The photoacoustic apparatus according to claim 21, wherein the signal processing unit is configured to obtain the effective attenuation coefficient using information on a distance from the position of the artery or vein inside the object to a light irradiation region on a surface of the object.

24. The photoacoustic apparatus according to claim 18, further comprising a display control unit configured to cause a display unit to display the sound pressure distribution information and a guide for the user to input the position of the artery or vein inside the object.

25. The photoacoustic apparatus according to claim 24, wherein the display control unit is configured to cause the guide to be displayed by extracting a position of a blood vessel in the object.

26. The photoacoustic apparatus according to claim 24, wherein the display control unit is configured to cause the guide to be displayed in a region facing the center of a light irradiation region on the surface of the object.

27. The photoacoustic apparatus according to claim 17, further comprising:
a light source configured to irradiate the plurality of lights having the plurality of wavelengths; and
a converting element configured to convert acoustic waves, generated from the object irradiated with the plurality of lights having the plurality of wavelengths, respectively, into electric signals, wherein
the signal processing unit is configured to obtain the plurality of sound pressure distribution information from the electric signals output from the converting element.

28. The photoacoustic apparatus according to claim 17, wherein the signal processing unit is configured to obtain the oxygen saturation at the position of the artery or vein inside the object which is instructed by an user.

29. A signal processing method for obtaining distribution information of oxygen saturation inside an object, using electric signals originated from acoustic waves which are generated from the object irradiated with a plurality of lights having a plurality of wavelengths, respectively,
the signal processing method comprising:
a step of obtaining a plurality of sound pressure distribution information originated from the plurality of lights having the plurality of wavelengths, respectively;
a step of obtaining position information on a position of an artery or vein inside the object,
a step of obtaining oxygen saturation at the position of the artery or vein inside the object represented by the position information; and
a step of obtaining the distribution information of the oxygen saturation in a region other than the artery or vein inside the object, using the plurality of sound pressure distribution information, the oxygen saturation at the position of the artery or vein inside the object, and information on absorption coefficient of oxyhemoglobin and de-oxyhemoglobin corresponding to each of the plurality of wavelengths.

30. The photoacoustic method according to claim 29, wherein the position information on the position of the artery or vein is obtained by instruction from an user.

31. The photoacoustic method according to claim 29, wherein the position information on the position of the artery or vein is obtained by performing image recognition.

32. A non-transitory computer-readable medium storing a program causing a computer to execute the signal processing method according to claim 29.
